(19) United States
(12) Patent Application Publication
(10) Pub. No.: US 2014/0029820 A1
(43) Pub. Date: Jan. 30, 2014

(54) DIFFERENTIAL GEOMETRIC METRICS CHARACTERIZING OPTICAL COHERENCE TOMOGRAPHY DATA

(71) Applicant: Carl Zeiss Meditec, Inc., (US)

(72) Inventors: Siddharth SRIVASTAVA, Antioch, CA (US); Homayoun BAGHERINIA, Oakland, CA (US)

(73) Assignee: CARL ZEISS MEDITEC, INC., Dublin, CA (US)

(21) Appl. No.: 13/744,911

(22) Filed: Jan. 18, 2013

Related U.S. Application Data
(60) Provisional application No. 61/589,162, filed on Jan. 20, 2012.

(51) Int. Cl. A61B 3/10

(52) U.S. Cl. CPC A61B 3/102
USPC 382/131

(57) ABSTRACT

A method is disclosed for analyzing 3D image data generated from optical coherence tomography (OCT) systems. The first step in the method is to identify one or more surfaces within the 3D data set. The surfaces are then characterized using geometric primitives. Geometric primitives such as concavities, convexities, planar parts, saddles, and crevices can be used. In a preferred embodiment, the primitives are combined. Various pathological conditions of the eye can be evaluated based on any analysis of the primitives.

### PRIORITY

This application claims priority to U.S. Provisional Application Ser. No. 61/589,162 filed Jan. 20, 2012, hereby incorporated by reference in its entirety.

### TECHNICAL FIELD

The present invention relates to medical imaging, and in particular data processing methods for data acquired through optical coherence tomography (OCT).

### BACKGROUND

Optical Coherence Tomography (OCT) is a technology for performing high-resolution real time optical imaging in situ. In Frequency Domain OCT (FD-OCT), the interferometric signal between reference light and the back-scattered light from a sample point is recorded in the frequency domain rather than the time domain. After a wavelength calibration, a one-dimensional Fourier transform is taken to obtain an A-line spatial distribution of the object scattering potential (A-scan). The spectral information discrimination in FD-OCT can be accomplished by using a dispersive spectrometer in the detection arm in the case of spectral-domain OCT (SD-OCT) or rapidly tuning a swept laser source in the case of swept-source OCT (SS-OCT). Laterally scanning the sample beam over a series of adjacent A-scans synthesizes cross-sectional images, creating a 2-D tomogram commonly called a B-scan. Typically, volumes are acquired by laterally scanning the sample beam over a series of B-scans; however alternative scan patterns, such as a spiral scan patterns, have been suggested to acquire OCT volume data.

OCT is widely used in ophthalmology to obtain high resolution images of the retina and of the anterior segment of the eye that can be used by doctors to view, diagnose and follow various pathologies in the eye over time. Generally, many closely spaced tomograms around the area of interest are arranged together in a cube/volume format. The histological composition of the retina is a layered arrangement of various tissue types, each having different optical properties relative to the wavelength of the laser used by the OCT device. Consequently, the image representation of the histological structure is a depth (spatially encoded) map of the optical properties of the area being imaged. Visually, the structure of the imaged retina is a curved and layered ordering of intensity. For a given wavelength, the relationship between the intensity and the tissue type it maps to is bound within a statistically defined range for a normal retina, and this specific relationship, along with the specific structure, forms the basis of both the interpretation and processing of the FD-OCT images. Retinal constitution of a typical eye, as imaged by OCT, consists of a layered stack of specific intensities, where each intensity represents a specific tissue type. For example, the nerve fiber layer (NFL) and the retinal pigment epithelium (RPE) appear as a bright layers, and the other layers are of significantly lower intensities.

A boundary between two different tissue types or layers is an important computational object, and can be automatically estimated as a set of discrete points by intensity based routines. This can subsequently be represented as a smooth surface fitted over the estimated set of points which best discriminate two specific intensity distributions in the image. A boundary, hence, is a smooth surface that separates two specific tissue types, and acts as a surrogate for the layers it separates. The spatial evolution of this boundary surface in space defines the anatomical structure of the retinal layer. Clearly, a boundary as derived above integrates both the intensity and structural information in a single mathematical object.

Pathologies of the eye often present as structural and intensity modifications of the affected area in the OCT images. Further, because of the functional specificity of the various layers of the retina, different pathologies may affect only a specific subset of the various layers, while sparing the rest of the retinal constitution. This results in a change in a spatial relationship between various retinal layers, and quantifying this change is often a good indicator of an evolving or developed ocular pathology.

One well established analysis technique, the RPE elevation analysis implemented in the commercially available Cirrus HD-OCT (Carl Zeiss Meditec, Inc. Dublin, Calif. and U.S. Pat. No. 7,668,342) fits the retinal pigment epithelium surface in order to approximate the expected broad shape of the eye so that deviations from that slowly changing fitted surface can be identified as elevations often associated with drusen secondary to dry age related macular degeneration. This method uses the layer which best separates the RPE-like intensities in the OCT image from the rest of the inner retina, as a base layer, and uses the Euclidean distance of another characteristic layer (like the ILM or RNFL boundary, estimated from intensity based segmentation routines) from this base layer to generate an elevation map. This elevation map can then be examined visually or fed into quantification routines to determine the deviation of the retinal structure from normality. The RPE fit requires 2 surfaces, and hence quantifies a relative measure across retinal layers. One limitation of this method is that the eye is not always well characterized by the function that is currently implemented. Another limitation is that local deviations that are not due to pathology, such as curvature changes related to the optic disc, may be mischaracterized as drusen. The method described in this document is capable of deriving diagnostically useful information for a single surface alone, although alternative embodiments can combine information from multiple boundaries to construct a more detailed view of the ocular pathology under investigation.

Since there is an implicit integration of structure and intensity during the interpretation of FD-OCT retinal images, automated methods for processing, interpretation and diagnosis of such images can benefit by an explicit extraction of the structure of the eye through the use of differential geometrical tools. The structural information can be used alone, or it can be used to append and enhance what can normally be achieved and understood by intensity alone. Similar types of analysis can also be performed for extra-retinal structures of the eye, for example, the anterior segment structures, including the cornea, crystalline lens and the iris.

### SUMMARY

It is an object of the present invention to use geometrical primitives to characterize various regions of 3D OCT image data of the eye. The method relies on intensity based routines to automatically identify regions of interest within a volume of OCT data. In the preferred embodiment of the present invention, representative axial locations of tissue within the eye representing pixels belonging to a specific retinal boundary or a collection of retinal tissue boundaries of interest are identified. With such a selection made for all axial columns in the entire image volume, one gets a set of data points distributed in the 3D volume, and the gestalt of the specific spread of points defines a specific boundary or surface. One skilled in the art may employ other approaches to get a classification of specific point localizers, which can be based on local searches, or volumetric approaches for classification based on intensity distribution in the entire imaged volume.

The choice of a specific boundary or boundaries for consideration can be determined by the pathology under examination or by input from the system's operator. An alternative embodiment of the invention derives an omnibus analysis of all retinal layers to permit a more exhaustive map of normality or retinal pathology based on geometric properties. A further alternative embodiment could use closed surfaces including the internal or external surface of a lesion or a retinal or choroidal vessel.

Once a set of points representing the tissue of interest has been obtained, a smooth surface can be interpolated over the points to yield a dense representation of the specific boundary as it varies across the area that has been imaged as illustrated in FIG. 1 for the retinal pigment epithelium (RPE). While only one B-scan is illustrated in the figure, data from multiple B-scans extending across the volume of data would be used to generate the smooth surface. This surface provides a mathematical model of the geometry of the tissue, and can be used to calculate local differential geometric properties of the tissue. As indicated in FIG. 2, the local differential properties can be combined to quantify the surface into regions (parcellations) having specific topographical structures in terms of common differential geometric primitives, i.e. concavities, convexities, bumps, dents etc. Knowledge from clinical practice indicates that many retinal pathologies have a visual appearance detectable in OCT images where specific layers appear deformed and distorted beyond their normal appearance. The invention described herein attempts to link these pathological deformations with differential geometric primitives to which they bear a close resemblance, and hence quantify, classify and localize the affected portion of the imaged retina. The generalized method and its application to a few pathological conditions will be described in detail below.

### BRIEF DESCRIPTION OF THE FIGURES

FIG. 1 illustrates a smooth surface fit to an identified retinal layer according to one aspect of the present invention.

FIG. 2 illustrates how different geometrical primitives can be used to characterize a retinal surface according to an aspect of the present invention.

FIG. 3 illustrates the steps of a generalized embodiment of the present invention.

FIG. 4 is a diagram of a generalized frequency-domain OCT system for use in ophthalmology. [delete “prior art” from Figure]

FIG. 5 illustrates how local curvatures can be determined on a surface on a point by point basis.

FIG. 6 illustrates the sequence of operations required to go from a surface to the geometrical primitives using various combinations of principal curvatures.

FIG. 7 illustrates an ordering of geometrical primitives based on a particular combination of principal curvatures or shape index according to one aspect of the present invention

FIG. 8 illustrates the retinal shape of a typical staphyloma case, showing the highly curved shape of the retinal features, including the RPE.

FIG. 9 illustrates local shape anomalies in the usual shape of the RPE and neighboring layers characteristic of PEDs.

FIG. 10 shows an OCT B-scan containing a serous PED.

FIG. 11 illustrates the layers of interest in the anterior segment that would be used to apply the present invention to the analysis of keratoconus.

### DETAILED DESCRIPTION

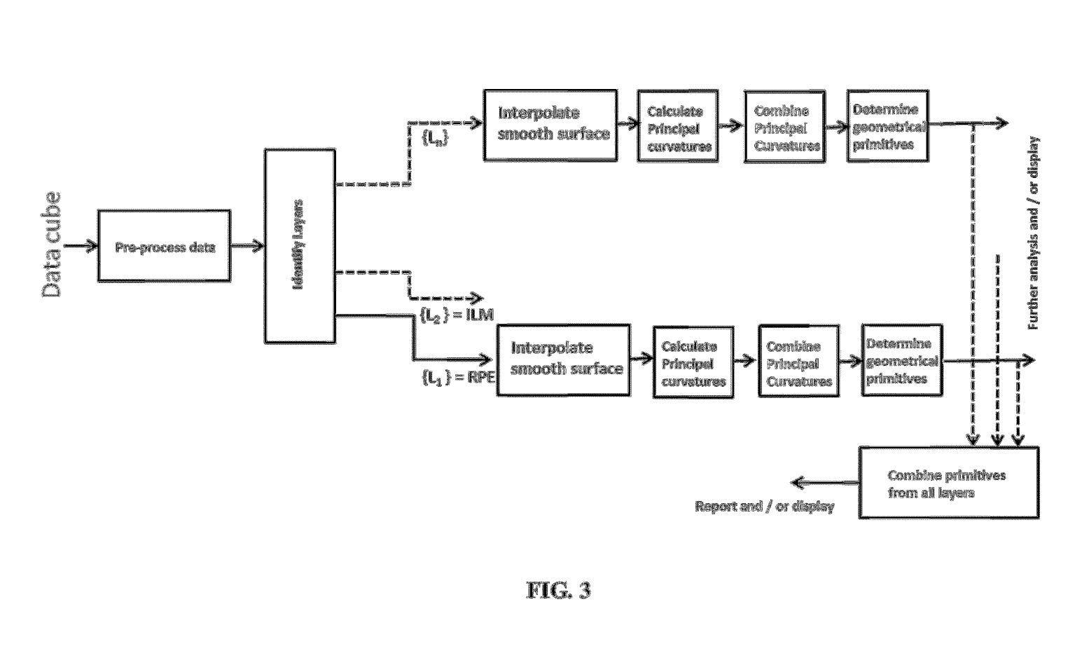

A flow chart illustrating a generalized embodiment of the present invention is shown in FIG. 3 for a collection of n retinal boundaries or surfaces. Dashed paths denote optional steps in the embodiment. Each layer boundary, Lₙ is interpolated and analyzed individually. After calculating the principal curvatures for a particular layer boundary, shape indices are designed from specific combinations of principal curvatures. These combinations can be used to classify portions of the surface into geometric primitives. The resulting geometrical primitives can be considered alone for a single surface or be combined to provide a more complete analysis of a tissue subvolume. The workings and capabilities of the method, and the construction of a pathology specific metric, can be illustrated by looking at specific disorders where differential geometric primitives can be used to model the visualized pathology.

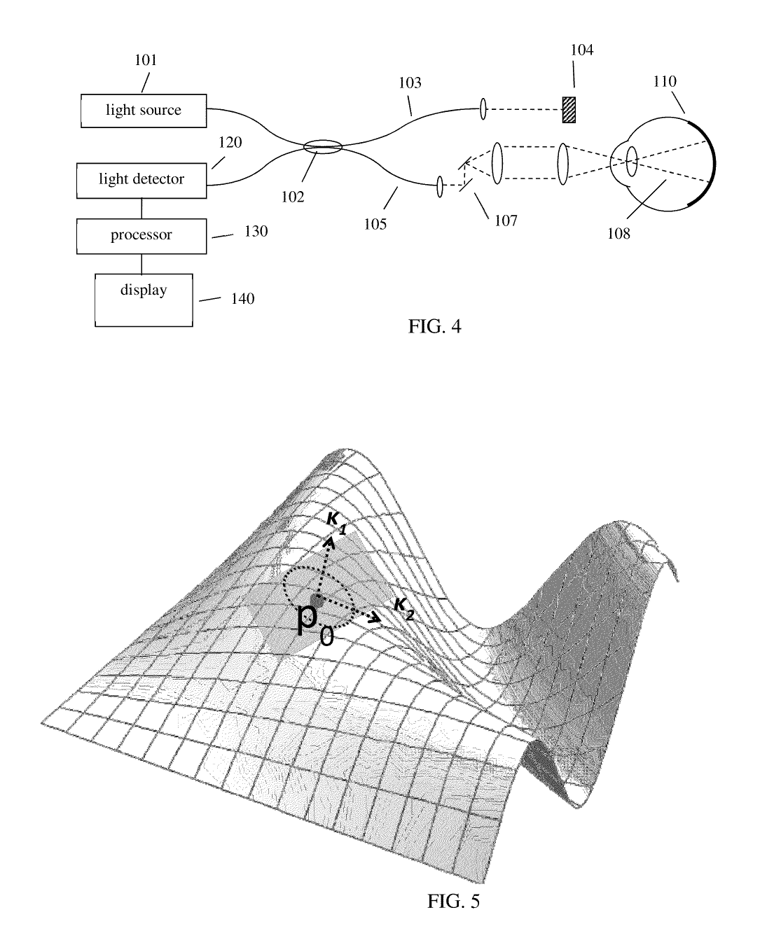

This method is intended to be used on image data obtained from a sample. The illustrations in this application are based on image data derived from a spectral-domain optical coherence tomography system (SD-OCT), but the methods could to other types of OCT data. A basic frequency-domain OCT (FD-OCT) system is illustrated in FIG. 4. Such an instrument generates 3D intensity data corresponding to an axial reflection distribution arising from reflecting features in the eye. As noted above, this information is currently used by doctors to view and diagnose various pathologies in the eye. The OCT system typically includes a spatially coherent source of light, 101. This source is typically either a broadband light source with short temporal coherence length or a swept laser source. (See for example, respectively, Wojtkowski, et al., [Ophthalmology 112(10):1734 (2005)] or Lee et al. [Optics Express 14(10):4403 (2006)].)

Light from source 101 is routed, typically by optical fiber 105, to illuminate the sample 110, a typical sample being tissues at the back of the human eye. The light is scanned, typically with a scanner 107 between the output of the fiber and the sample, so that the beam of light (dashed line 108) is scanned over the area or volume to be imaged. Light scattered from the sample is collected, typically into the same fiber 105 used to route the light for illumination. Reference light derived from the same source 101 travels a separate path, in this case involving fiber 103 and retro-reflector 104. Those skilled in the art recognize that a transmissive reference path can also be used. Collected sample light is combined with reference light, typically in a fiber coupler 102, to form light interference in a detector 120. The output from the detector is supplied to a processor 130. The results can be stored in the processor 130 or displayed on display 140.

The interference causes the intensity of the interfered light to vary across the spectrum. For any scattering point in the sample, there will be a certain difference in the path length between light from the source and reflected from that point, and light from the source traveling the reference path. The interfered light has an intensity that is relatively high or low depending on whether the path length difference is an even or odd number of half-wavelengths, as these path length differences result in constructive or destructive interference respectively. Thus the intensity of the interfered light varies with wavelength in a way that reveals the path length difference; greater path length difference results in faster variation between constructive and destructive interference across the spectrum. The Fourier transform of the interference spectrum reveals the profile of scattering intensities at different path lengths, and therefore scattering as a function of depth in the sample [see for example, Leitgeb et al, Optics Express 12(10):2156 (2004)]. Typically, the Fourier transform results in complex data, and the real part of the results are tabulated to construct the intensity image. The imaginary part encodes information related to the phase shifts arising from local sample motion, and can be used to deduce quantities related to physical motion of dominant scatterers in the sample.

The profile of scattering as a function of depth is called an axial scan (A-scan). A set of A-scans measured at neighboring locations in the sample produces a cross-sectional image (tomogram) of the sample. A series of tomograms can be combined into a 3D image cube. Various tissue layers can be identified and segmented in the 3D image volume using techniques well known in the field.

The determination of the set of data points representing the region of tissue to be analyzed from the 3D volume of data can be performed by a multitude of methods. The choice essentially depends on the specific contrast of the retinal layer of interest in relation to the intensity levels in its immediate vicinity. Towards this end, in the preferred embodiment, intensities are examined column wise (axially), and a local measure of the average intensities are generated, and mapped to specific boundaries or layers based on some prior knowledge of typical intensity distributions of landmark retinal layers, like the RPE. Specifically, the RPE can be localized as points in A-scans having the highest intensities. In another embodiment, in the case where the pathology disrupts the reflectivity of layers, one can also include the fast and slow scan directions (volumetric methods) to construct a statistical measure of the intensity distribution that takes into account the intensities of neighboring points in the axial, fast and slow directions. If the pathology is not too widespread, information from neighboring points spared by the pathology may increase the confidence with which the affected layer can be localized. In another embodiment of the present invention, a coarse to fine classification of the retina may be performed in parts, based on intensity and higher order statistics of the intensity distribution, using standard tools in statistics (histogram, joint distributions etc.). In the case of all embodiments, a set of points co-located in the 3D data volume will result which will describe the position and the shape of the boundary of interest.

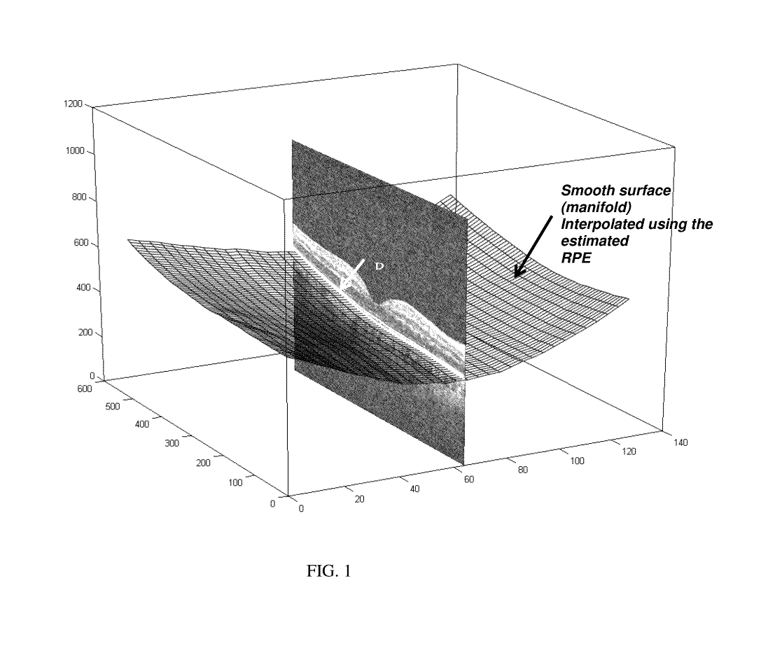

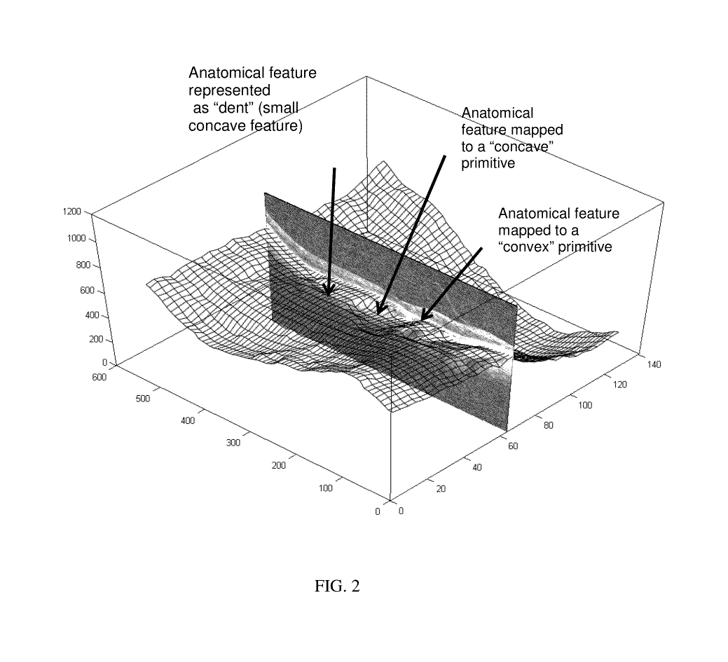

Once a representative set of points has been obtained for all the layers being analyzed, the inventive method proceeds by interpolating a smooth surface over this point set as shown in FIG. 1 for the RPE. Since the point set may only sparsely identify the location of a specific boundary in the volume, interpolation works by filling in the gaps between these points separated by the imaging resolution in the fast (x-resolution) and the slow B-scan (y-resolution) directions to yield a more dense representation. These gaps can be filled by estimating a best fitting Nth order polynomial, given the known data values on a regular grid defined by the x and y resolution. Those skilled in the art can use popular methods like 2D Splines or Biezer curves to fit an arbitrary order polynomial to the known data values. The advantage of transforming from a point set representation to a surface model is two-fold: first, a surface, being a dense representation, enables higher sampling, and allows the method to generate results at sub-pixel locations with a sub-pixel accuracy. Second, since our local metrics are essentially differential operators, a smooth surface avoids discontinuities (other than that imposed by the pathology) and hence avoids singularities in the solution.

After a smooth surface has been constructed, the preferred embodiment of the inventive method then proceeds by calculating principal curvatures locally over the entire surface as will be elucidated in the following steps. Let [x,y] be the parameterization of the surface, and let S[x,y] represent the smooth surface. It should be mentioned here that the surface is a two dimensional entity embedded in the three dimensional space of the OCT volume. Hence, only two coordinates are sufficient to span its entire extent.

With ∂_{i,j} representing the partial derivative with respect to variable j, followed by i, a preferred embodiment calculates the hessian H:

$$H = ( \begin{matrix}{\partial }_{x , x} S & {\partial }_{x , y} S \\ {\partial }_{y , x} S & {\partial }_{y , y} S\end{matrix} ) = ( \begin{matrix}{S}_{xx} & {S}_{xy} \\ {S}_{yx} & {S}_{yy}\end{matrix} ) \qquad ( EQN 1 )$$

The two eigenvalues of H are given by:

$$\begin{matrix}\frac{1}{2} ( {S}_{xx} + {S}_{yy} - \sqrt{{S}_{xx}^{2} + 4 {S}_{xy} {S}_{yx} - 2 {S}_{xx} {S}_{yy} + {S}_{yy}^{2}} ) = {k}_{1} & ( EQN 2 A ) \\ \frac{1}{2} ( {S}_{xx} + {S}_{yy} - \sqrt{{S}_{xx}^{2} + 4 {S}_{xy} {S}_{yx} - 2 {S}_{xx} {S}_{yy} + {S}_{yy}^{2}} ) = {k}_{2} & ( EQN 2 B )\end{matrix}$$

These eigenvalues of the Hessian at every point on the surface quantify the maximum and minimum rate of change of the surface along two orthogonal directions respectively at each point on the surface, and are oriented in the directions given by the eigenvectors of the Hessian at the same point. The directions represented by the eigenvectors are called the principal directions at the point of interest, and the eigenvectors are the principal curvatures. The orthogonal directions together form a reference frame at each point, and are dependent on the local shape of the surface at that point.

A schematic illustrating the abovementioned concepts is given in FIG. 5. Principal directions are calculated on a surface at point p₀.K₁ and k₂ are the maximum and minimum curvatures of the surface in the local neighborhood of p₀. These principal curvatures are calculated at all points on the surface, and completely characterize how the surface evolves in space. The complete rate of surface change at each point is represented with respect to this local coordinate frame at that point, over the entire surface. These principal curvatures are intrinsic representations of the local variations on the surface, and as will be described in further detail below can be algebraically combined in various ways to generate a multitude of values at each point, which together can quantify various properties of the local shape of the surface. Each algebraic combination which can reveal, highlight or discriminate a surface feature of interest from all other geometric features of the surface, is a potential geometrical primitive. The combinations can be used to construct a 2D map of shape descriptors for each case under examination, and such maps from all cases (or all visits) can be combined to generate a cross-sectional (or longitudinal) map of the disease, which can be used to examine patterns for discrimination, or track disease progression. In one embodiment, principal curvatures can be combined to generate a single value at each point (metric), resulting in a dense map of the metric over the entire surface. Similar anatomical features will have similar values in this new map, hence the surface can be parcellated into similar geometrical regions by examining the histogram of the metric, followed by a threshold operation at interesting points. The relative proportion and spatial distribution of these specific geometrical regions can be combined into one omnibus metric to quantify the pathology. For example, for posterior staphyloma (the imaging hallmark of which is a highly curved retina), one can partition the RPE surface into planar and concave regions, and the relative proportion of planar and concave regions can be combined to construct a metric sensitive to highly curved retinas. (See Illustrative case 1 below). Metrics developed in this way, or a set of primitives identified in this way, could be compared for an individual patient to the range of metrics observed in a database of normal eyes, or even to a database of abnormal eyes, where the abnormal eyes could be specific to a disease of interest or to a disease, such as staphyloma, known to have certain geometric characteristics.

FIG. 6 illustrates how the principal curvatures of a surface can be combined mathematically in four different ways to determine geometrical primitives of the surface. The combinations of the principal curvatures can be constructed to map each point to a 2D graph with each point of the 2D graph representing a possible variation of the local shape. The right column illustrates four primitives that are obtained from an algebraic combination of the principal curvatures at each point on the surface shown in the left panel. Depending on how the principal curvatures are combined, different features of the surface are highlighted. Each algebraic combination of the principal curvature is hence, a geometrical primitive for the specific surface feature that it helps to highlight. Similar anatomical developments, for example, PEDs will have similar shape, and hence will cluster together in a resulting 2D map (see Illustrative Case 2 below). Furthermore, similar quantifications from multiple boundaries can be co-mapped to the same 2D graph to look for inter-layer clustering of geometry for a deeper understanding of how a specific pathology effects the bulk of the retinal tissue (for example, mass effect due to an occult lesion). Further extensions of the method may stack these 2D maps of longitudinal or cross-sectional data to evaluate progression in this alternative space. Those skilled in the art may realize that the specific combination of local curvature measurements has to be pathology specific for the method to have good identifying or discriminating power.

The geometrical primitives can be combined and quantified based on a numerical scale or shape index for specific layers or areas of interest within the 3D volume. One of the combinations of principal curvatures, illustrated in FIG. 6, is.

$$s = \frac{2}{π} arctan \frac{{κ}_{2} + {κ}_{1}}{{κ}_{2} - {κ}_{1}} \\ {κ}_{1} \geq {κ}_{2} \qquad EQN 3$$

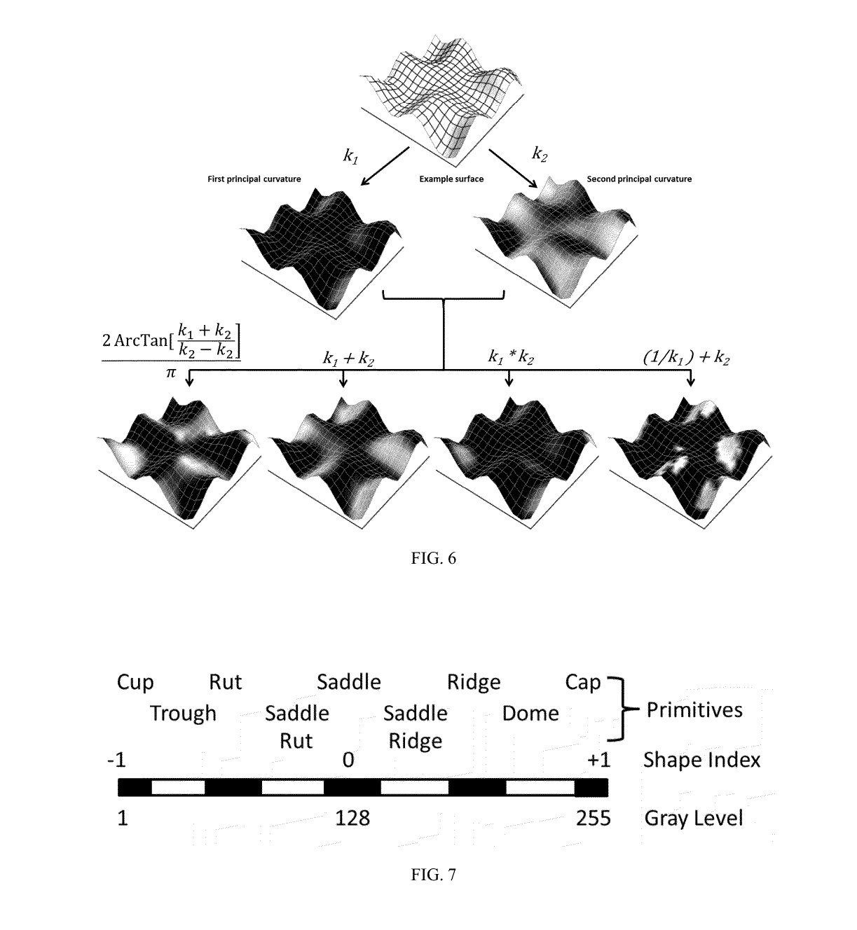

This combination generates an ordering of geometric primitives on a scale of [−1, 1], as illustrated in FIG. 7. This ordering allows a thersholding based on the value of the transverse location.

As will be described in Illustrative Case 5 below, the method presented herein is not confined to the posterior segment of the eye, but can be used for anterior segment imaging as well. In this case one would get a point set representing the epithelial, endothelial or the medial stroma layer for the cornea for example, and proceed with the subsequent calculation as detailed in the preceding description.

One skilled in the art can appreciate that the general method of the invention could be applied to additional areas in which it would be useful to automatically characterize pathologies characterized to geometric properties of a specific layer or group of layers in the eye. Furthermore, recent trends and future development in OCT imaging may provide images with different intensity distribution and characteristics for each specific layer, but the essential geometric structure of the retinal layers would remain the same. To make the methods compatible to the new imaging data, intensity transformation techniques, like scaling, stretching or histogram equalization can be used to normalize the data to a common scale, which can then be used for computations and derivations as described herein.

The invented method will now be described in detail below as it relates to three specific applications in the field of ophthalmology.

### Illustrative Case 1: Staphyloma

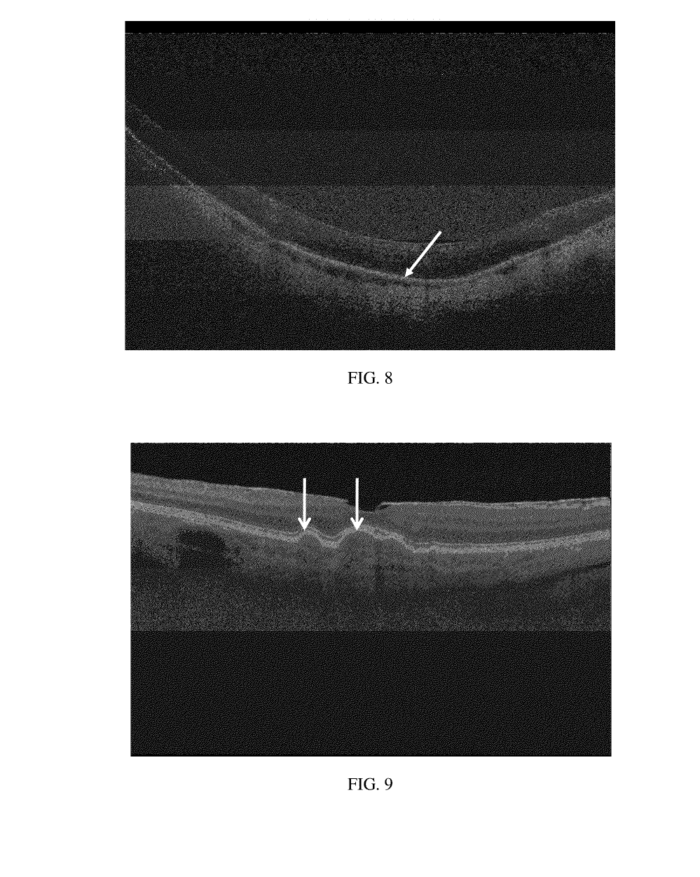

A staphyloma is an abnormal protrusion of the uveal tissue through a weak point in the eyeball, and as such it may be characterized by an abnormal concavity in the tissue being imaged. The degree of this concavity is strongly correlated to the severity of the disorder. Diagnostically, it makes sense to examine the disorder as a function of some metric dependent on tissue curvature. For posterior staphyloma it makes sense to look at the curvature of the retinal tissue. As illustrated in FIG. 8, the curvature of the Retinal Pigment Epithelial (RPE) layer can be a good indicator of the curvature of the retina for this case. The arrow points to the RPE layer.

An added benefit of using the RPE is that, in most cases, this layer can be segmented with appreciable accuracy. Hence it provides a robust point set to mathematically interpolate a smooth surface through, and then use this defining surface to calculate its local differential geometry at each point. One skilled in the art would appreciate that alternative layers or additional layers could be grouped with the RPE and used for characterizing retinal curvature. Furthermore, because the eye is in general roughly spherical, any intensity based method for identifying the 3D position of points representative of any tissue in the eye might be used to characterize the curvature and relate that to pathology when such pathology results in a distortion from the usual shape.

In the preferred embodiment of the present invention, the highly curved shape of the retina in cases with staphyloma is exploited directly to design a metric related to the atypical retinal shapes associated with the disorder from the typical cases. Such a metric can be correlated to severity for the purposes of staging, or used to discriminate normal from pathological retinas. Software used in the Cirrus OCT instrument (Carl Zeiss Meditec Inc. Dublin, Calif.) automatically generates points representing the RPE layer as well as a smooth fourth order polynomial to designate a fit to the RPE layer. The metric of the present invention described herein exploits the geometry of the RPE layer to characterize the curvedness of the retina, but instead of taking the polynomial specifying the RPE, it fits a quadratic (second order in x and y coordinates) surface. Using a quadratic illustrates the preferred embodiment of the pathology specific metric, and ensures that the resulting surface will essentially be made up of two primary geometrical features, planar, and concave. Also, since the primary objective is to derive a global measure signifying curvedness, fitting a lower order surface also helps in reducing the effect of small scale undulations in the RPE surface from contributing to the global estimation. After fitting the quadratic surface to the points in the least-squares sense, principal curvatures over the surface were calculated, which give, at each point pⱼ of the surface, a pair of values indicating the maximum (kₘₐₓ) and minimum (kₘᵢₙ) change in curvature at that point. These were combined at each point to yield r[pⱼ]=√(kₘₐₓ²+kₘᵢₙ²)/2. Generally, values of r<0.05 are indicative of a local geometry that is essentially planar, and curved parts of the surfaces have values 0.05 or larger. To generate the final quantification of curvedness, let N=total number of points on the surface, {p₁, p₂ . . . pₙ} be the set of N_{c} points where r>=0.05, and S_{c}=Σr [pⱼ], j=1 . . . n, then

p_{concave}=(N_{c}*S_{c})/N EQN 4

The set of points or locations for which this analysis is performed could be the full set of points that represent the surface, or they could be a subset of locations that represent small local areas. In the limiting case the curvedness could be defined for the entire surface.

The metric p_{concave} combines both the proportion of points that are undergoing local changes in curvature (N_{c}/N), and also the total magnitude of this change (S_{c}), and hence has sensitivity for a large range of retinal curvedness patterns associated with staphyloma. The metric p_{concave} also illustrates the preferred embodiment of selecting specific geometrical primitives best featured for the problem at hand. In staphylomas, the most informative geometrical primitive is a concave shape, and the relative proportion of planar and concave developments of the retinal relate to the severity of the disease.

Alternatively, if we select points where r<=0.05, we get pₚₗₐₙₐᵣ which gives high values if the surface has a higher proportion of planar sections throughout its extent.

In an alternative embodiment, kₘᵢₙ and kₘₐₓ at each location, for each separate case, can be mapped to a 2D graph spanned by principal curvature axes. This representation can be used to either track disease progression over time by mapping each time point data to the initial reference graph, or to look at cross-sectional data by mapping several subjects on the same graph.

### Illustrative Case 2: PED

Pigment epithelial detachment (PED) cases typically show shape anomalies in the usually smoothly varying structure of the RPE and neighboring layers. As illustrated in FIG. 9, the changes in shape are usually dome shaped elevations along the RPE, which can occur with varying frequency and height above the RPE. In another embodiment of the technique of the present invention, these local shape variations can be modeled by local curvature measurements performed on the RPE layer followed by the classification of this curvature map into regions of concave, convex or planar features. This directly provides a localization of the anomalous portions of the RPE, and also helps quantify the specific pathology on a case by case basis.

### Illustrative Case 3: Serous PED

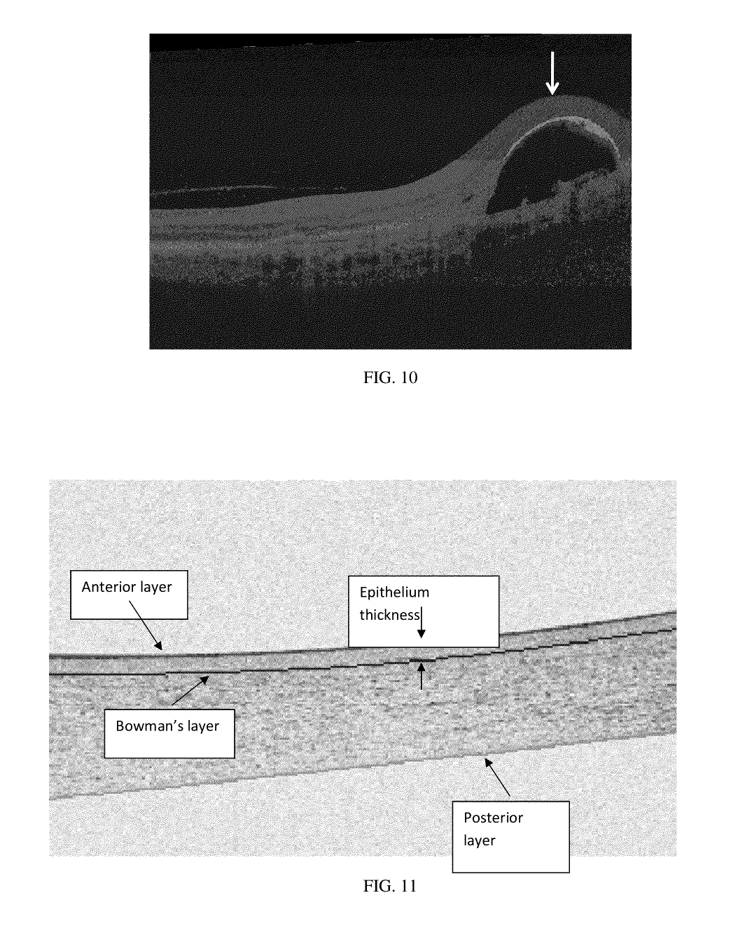

This case shows an example of a serous PED, marked by a hyper-elevation of the hyper-reflective RPE, in conjunction with a loss in reflectivity in retinal tissue underlying the location of the visible elevation as illustrated in FIG. 10. There is a shaped malformation of the RPE (brightest band) and the ILM (indicated by arrow) assumes a similar shape. The differential geometric analog of this pathology is clearly a dome shaped geometrical primitive, which can be mathematically represented and localized in terms of principal curvatures evaluated on a surface representing the RPE (Retinal pigment epithelium). Also noteworthy is that for this case, the mass effect of the pathology causes a similar geometrical change in the innermost layer as well (the inner limiting membrane, ILM, pointed to by the white arrow), and all the visible layers in the intermediate retinal tissue. Hence, both the RPE and ILM can be examined simultaneously to localize this effect, although analysis of either layer may be sufficient for the illustrative case.

### Illustrative Case 4: Kerataconus

Keratoconus is a corneal disorder that presents later in the disease process with central corneal thinning, corneal protrusion and progressive irregular astigmatism. The structural changes within the cornea cause the corneal shape to be more a conical shape than a normal gradual surface. The detection of early keratoconus is one of the major safety issues faced in corneal refractive surgery, as performing LASIK on undiagnosed keratoconus has been identified as the leading cause of ectasia after laser refractive surgery.

The OCT epithelial thickness map can be used to detect early keratoconus since local epithelial thickening is caused by keratoconus. The OCT epithelial thickness map has greater local curvatures than pachymetry maps and can be used as an input for a detection algorithm.

Here a model-based method for early keratoconus detection is presented. It will require a certain number of training data sets for keratoconus cases. A large set of training data collection for keratoconus cases is time consuming and very expensive. Thus, one advantage of this method will be that a limited number of training data is required to model the keratoconus class. A data-driven classifier based method is not practical since it requires a large number of keratoconus cases to ensure the robustness of the detection.

The method can be accomplished in several ways, either using geometric primitives as described above, or in a similar approach using Zernike polynomials or a Fourier series expansion. In either case, the method offers a solution to represent a 3-D arbitrarily complex continuous surfaces when the underlying surface is relatively smooth. In one embodiment, the Zernike polynomials will be used to fit the curvature map of the epithelial thickness map. The Zernike polynomials or geometric primitives of the keratoconus training data set will model the keratoconus class. An alternative method to Zernike polynomials is Fourier series expansion to model the curvature map of the epithelial thickness map. Other methods can be envisioned by one skilled in the art.

The detection algorithm can be summarized by one of the two following pairs of steps:

### Training—Geometric Primitives

Segment the epithelium layers of available keratoconus cases
Generate the epithelial thickness maps
Compute the geometric primitives associated with the epithelial thickness map
Build a subspace-based classifier

### Detection

Segment the epithelium layers of a given case
Generate the epithelial thickness map
Compute the curvature map of the epithelial thickness map
Compute the geometric primitives associated with the epithelial thickness map
Detection
   Training—Other mathematical fitting
Segment the epithelium layers of available keratoconus cases
Generate the epithelial thickness maps
Compute the curvature maps of the epithelial thickness maps
Compute the Zernike or Fourier representation of the curvature maps
Build a subspace-based classifier based on Zernike or Fourier coefficients

### Detection

Segment the epithelium layers of a given case
Generate the epithelial thickness map
Compute the curvature map of the epithelial thickness map
Compute the Zernike or Fourier representation of the curvature map
Detection

A general model that describes keratoconus cases is built based on a set of training data (scans of keratoconus cases). The detection uses this model to identify keratoconus cases. The detection could alternately use a model based on normal eyes to identify more generic deviations from normals.

### Epithelium Segmentation

The epithelium segmentation can be done using anterior segmentation and approximate epithelial thickness as prior knowledge which define the region of interest (ROI) for the segmentation algorithm. The ROI is used to find actual layer position (Bowman's layer) by utilizing the hybrid graph theory and dynamic programming framework. The layers of interest are illustrated in FIG. 11.

### Epithelial Thickness Map

A grid fitting or an interpolation method can be used to generate a complete 2-D epithelial thickness surface if a sparse or meridional scan pattern is used.

### Curvature Map

The mean curvature map of the 2-D epithelial thickness surface represents the local variation of the epithelial thickness surface. It is expected that this curvature map is a smooth map which can be modeled by Zernike polynomials or Fourier series.

### Map Model

In this invention, the Zernike polynomials or Fourier series will be used to fit the curvature map of the epithelial thickness map. The Zernike polynomials or Fourier series coefficients will be the features used to build a classifier.

### Classifier

Let the d-dimensional vector cⁱ=[d₁i, . . . , c_{d}ⁱ]^{T} represent the Zernike polynomials or Fourier series coefficients (of the curvature map of the epithelial thickness map) of case i, and let W be the linear space spanned by c⁽ⁱ⁾ over varying keratoconus cases. It is assumed that the vector cεR^{d} is constrained to live in a subspace V of dimension q<d. This is due to the fact that certain coefficients will be similar for different keratoconus cases.

This detection algorithm begins by modeling the subspace V for keratoconus cases. The Zernike polynomials or Fourier series coefficients of N keratoconus cases create the matrix Z=[c¹ . . . c^{N}] of d×N. A subspace of suitable dimension q is built from these observations via Principal Component Analysis by computing the SVD of matrix Z([u,s,v]=svd(Z), where s is a diagonal matrix of the same dimension as Z and contains the singular values of Z with nonnegative diagonal elements in decreasing order. The columns of u represent the basis of space W). It is expected that this procedure produces a subspace modeling based on available keratoconus cases (training data). The subspace dimension q can be determined based on the part of variance σ² (q) explained by first q axes (e.g. >t=0.9)

$$diag ( s ) = [ {λ}_{1} , … , {λ}_{d} ]$$

$${max}_{i} ( \frac{{{\sum }_{k = 1}}^{i} {λ}_{k}}{Trace ( s )} ) > ti ∈ [ 1 , … , d ]$$

The subspace dimension q can be also determined via cross-validation. For each subspace dimension, we compute the probability of correct detection (true positive) as follows. We run k rounds of cross-validation, each time selection 50% of training data at random, learning the subspace, and testing on the remaining 50% of training data. Then, we count the number of times any case is correctly detected, and divide the result by the number of cross-validation rounds (k) and by the number of cases. Other available data from different classes such as normal or post-LASIK, etc. can be used to estimate the probability of incorrect detection (false positives). A ROC curve for different design parameters (subspace dimension) helps us to choose the best subspace dimension (q).

The detection algorithm is based on a one-class classifier model: it analyzes a given vector c to determine whether or not it may belong to the keratoconus class, without explicitly modeling other classes such as normal or post-LASIK, etc. The reason for choosing a one-class classifier is that it would be very difficult to produce a general model of others classes with limited number of cases for different classes.

### Detection

Since the coefficients are expected to live in a subspace, a distance-based classifier could be obtained by thresholding the square distance of a given vector c (representing the Zernike polynomials or Fourier series coefficients) to such subspace V.

d(c,V)²=c^{T}c−c^{T}QQ^{T}c<τ

The columns of Q contain the orthonormal basis of the subspace of dimension q (the first q columns of u).

The threshold τ can be learned from the training data. The choice of threshold based on a limited number of training data is often too conservative, in which case we can multiply the threshold by a constant that can be determined using all keratoconus and other available cases.

The following references are hereby incorporated by reference:

### Patent Literature

US Patent Publication No. 2010/0111373 Chin et al. “mean curvature based de-weighting for emphasis of corneal abnormalities.
U.S. Pat. No. 7,779,641 Bille “Finite element model of a keratoconic cornea”
U.S. Pat. No. 7,497,575 Huang et al. “Gaussian fitting on mean curvature maps of parameterization of corneal ectatic diseases”
U.S. Pat. No. 7,668,342 Everett et al. “Method of bioimage data processing for revealing more meaningful anatomic features of diseased tissues”

### Non Patent Literature

J. J. Koendrink et al. “The structure of relief” Advances in Imaging and Electron Physics 103:65 1998.

J. J. Koendrink et al. “Two-plus-one-dimensional differential geometry” Pattern Recognition Letters 15(5): 439 1994.
Satoh N et al. “Accommodative changes in human eye observed by Kitasato anterior segment optical coherence tomography” Jpn J. Ophthalmol. 2012 Nov. 21.
Ortiz S et al. “In vivo human crystalline lens topography. Biomed Opt Express. 2012 Oct. 1; 3(10):2471-88.
Ohno-Matsui K et al. “Intrachoroidal cavitation in macular area of eyes with pathologic myopia” Am J. Ophthalmol. 2012 August; 154(2):382-93.
Wu W C et al. “Visual acuity, optical components, and macular abnormalities in patients with a history of retinopathy of prematurity” Ophthalmology. 2012 September; 119(9): 1907-16.
Baroni M et al. “Towards quantitative analysis of retinal features in optical coherence tomography” Med Eng Phys. 2007 May; 29(4):432-41.
Kinori M et al. “Enhanced S-cone function with preserved rod function: a new clinical phenotype” Mol Vis. 2011;17:2241-7.
Ikuno Y et al. “Ocular risk factors for choroidal neovascularization in pathologic myopia” Invest Ophthalmol Vis Sci. 2010 July; 51(7):3721-5.

What is claimed is:

1. A method to analyze pathological conditions in an eye based on a 3D optical coherence tomography (OCT) data set, said method comprising:
identifying one or more surfaces within the 3D OCT data set;
characterizing the one or more surfaces using one or more geometric primitives; and
displaying or storing the resulting geometric primitives.

2. A method as recited in claim 1, wherein the surface is identified using a layer segmentation of the 3D data set.

3. A method as recited in claim 2, wherein the segmentation identifies the RPE layer.

4. A method as recited in claim 1, wherein the geometric primitives are selected from the group consisting of concavities, convexities, planar parts, saddles, and crevices.

5. A method as recited in claim 1, further comprising comparing the calculated geometric primitives to primitives generated from image data for a collection of normals.

6. A method as recited in claim 1, wherein the displaying of the geometrical primitives is limited to specific primitives.

7. A method as recited in claim 6, wherein the limiting of the geometrical primitives is accomplished based on input from the user.

8. A method as recited in claim 1, wherein the step of characterizing includes interpolating the one or more surfaces, calculating principal curvatures for the one or more surfaces, and combining the principal curvatures in one or more ways to discriminate the geometric primitives.

9. A method as recited in claim 1, wherein the step of characterization includes subtracting one surface from the other to create a thickness map and characterizing the thickness map using one or more geometric primitives.

10. A method as recited in claim 1, further comprising comparing the calculated geometric primitives to primitives generated from image data for a collection of patients with a specific disease.

11. A method as recited in claim 1, wherein the pathological condition is one of staphyloma, keratoconus, or pigment epithelial detachment (PED).

12. A method to analyze pathological conditions in an eye based on a 3D optical coherence tomography (OCT) data set, said method comprising:
identifying one or more surfaces within the 3D OCT data set;
generating one or more geometric primitives that characterizes the identified one or more surfaces;
using the generated geometric primitives to identify structural abnormalities in the eye of a patient; and
displaying or storing information regarding the identified structural abnormalities.

13. A method as recited in claim 12, wherein the surface is identified using a layer segmentation of the 3D data set.

14. A method as recited in claim 13, wherein the segmentation identifies the RPE layer.

15. A method as recited in claim 12, wherein the geometric primitives are selected from the group consisting of concavities, convexities, planar parts, saddles, and crevices.

16. A method as recited in claim 12, further comprising combining multiple geometric primitives into a single metric.

17. A method as recited in claim 16, wherein the combination of geometric primitives used in creating the single metric is based on input from the user.

18. A method as recited in claim 16, wherein the single metric quantifies the severity of PEDs and is given by the number of convex regions multiplied by the average area of the convex regions divided by the average are of the planar regions.

19. A method as recited in claim 12, further comprising comparing the calculated geometric primitives to primitives generated from image data for a collection of normals.

20. A method as recited in claim 12, further comprising comparing the calculated geometric primitives to primitives generated from image data for a collection of patients with a specific disease.

21. A method as recited in claim 12, wherein the pathological condition is one of staphyloma, keratoconus, or pigment epithelial detachment (PED).

22. A method as recited in claim 12, wherein the frequency of convex geometric primitives can be used to identify PEDs.

23. A method as recited in claim 12, wherein the step of characterizing includes interpolating the one or more surfaces, calculating principal curvatures for the one or more surfaces, and combining the principal curvatures in one or more ways to discriminate the geometric primitives.

24. A method as recited in claim 12, wherein the step of characterization includes subtracting one surface from the other to create a thickness map and characterizing the thickness map using one or more geometric primitives.

25. A method to characterize a feature of an eye based on a 3D optical coherence tomography (OCT) data set, said method comprising:
identifying one or more surfaces using the 3D OCT data set;
generating one or more geometric primitives that characterizes the identified one or more surfaces;
using the generated geometric primitives to characterize the feature of the eye of a patient; and
displaying or storing information regarding the characterized feature.

26. A method as recited in claim 25, wherein the surface is identified using a layer segmentation of the 3D image volume.

27. A method as recited in claim 26, wherein the segmentation identifies the RPE layer.

28. A method as recited in claim 25, wherein the surface is identified using a quadratic fit.

29. A method as recited in claim 25, wherein the geometric primitives are selected from the group consisting of concavities, convexities, planar parts, saddles, and crevices.

30. A method as recited in claim 25, further comprising combining multiple geometric primitives into a single metric.

31. A method as recited in claim 30, wherein the combination of geometric primitives used in creating the single metric is based on input from the user.

32. A method as recited in claim 18, wherein the characterization involves comparing concave primitives to planar primitives to identify cases of myopia.

33. A method as recited in claim 18, further comprising comparing the calculated geometric primitives to primitives generated from image data for a collection of normals.

34. A method as recited in claim 18, further comprising analyzing topological deviations in addition to the geometric primitives to determine gross structural anomalies.

35. A method as recited in claim 18, wherein the step of characterizing includes interpolating the one or more surfaces, calculating principal curvatures for the one or more surfaces, and combining the principal curvatures in one or more ways to discriminate the geometric primitives.
